(19) United States
(12) Patent Application Publication
(10) Pub. No.: US 2014/0295542 A1
(43) Pub. Date: Oct. 2, 2014

(54) CELL SEPARATION COMPOSITIONS AND METHODS FOR SEPARATING AND RECOVERING THERAPEUTIC CELLS IN BLOOD TISSUE

(71) Applicant: BHC Technology Holdings LLC, St. Paul, MN (US)

(72) Inventor: Daniel Patrick Collins, Lino Lakes, MN (US)

(73) Assignee: BHC Technology Holdings LLC, St. Paul, MN (US)

(21) Appl. No.: 14/230,679

(22) Filed: Mar. 31, 2014

Related U.S. Application Data
(60) Provisional application No. 61/807,167, filed on Apr. 1, 2013.

(51) Int. Cl. C12N 5/0783

(52) U.S. Cl. CPC C12N 5/0636
USPC 435/325; 252/60

(57) ABSTRACT

Embodiments provide a cell separation composition and methods for separating and recovering desired therapeutic cells in a blood tissue sample.

### RELATED APPLICATIONS

This application claims priority to U.S. provisional application No. 61/807,167, the entire contents of which are incorporated herein by reference.

### FIELD OF THE INVENTION

The present invention generally relates to cell separation compositions and methods for separating and recovering therapeutic cells in a blood tissue sample.

### BACKGROUND OF THE INVENTION

Many medical procedures involve the removal of blood tissue from a donor and the subsequent introduction of that tissue into a recipient. Blood tissue includes but is not limited to peripheral blood, umbilical cord blood, menstrual blood, bone marrow, spleen tissue and lymphatic tissue. Cells in blood tissue have been shown to have variety of therapeutic benefits in recipients. However, before introducing donor blood tissue into a recipient, the blood tissue must be screened to determine if the cells in the tissue are likely to be accepted or rejected by the recipient.

One common screening procedure used is ABO blood type screening. Red blood cells (erythrocytes) have different surface antigens that produce different antibodies and cause individuals to have different blood types. There are four principal blood types: A, B, AB, and O. There are two antigens and two antibodies that are mostly responsible for the ABO types. The specific combination of these four components determines an individual's blood type.

Individuals with type A blood will have the A antigen on the surface of their red cells. As a result, they do not produce anti-A antibodies because these antibodies would cause the destruction of their own blood. However, they do produce anti-B antibodies. If B type blood is injected into their systems, anti-B antibodies will recognize it as foreign and burst or agglutinate the B-type red cells in order to cleanse the blood of foreign protein. Likewise, individuals with type B blood will have the B antigen and do not produce anti-B antibodies but do product anti-A antibodies. If A type blood is injected into their systems, anti-A antibodies will recognize it as foreign and burst or agglutinate the A-type blood cells.

Individuals with type O blood do not produce ABO antigens. Therefore, their blood normally will not be rejected when it is given to others with different blood types. As a result, type O people are universal donors for blood transfusions. On the other hand, type O blood produces both anti-A and anti-B antibodies. As such, individuals with type O blood can receive only type O blood themselves. Those with type AB blood have both the A antigen and B antigen on the surface of their red cells and do not make any ABO antibodies. Their blood does not discriminate against any other blood type. Consequently, they are universal receivers for transfusions. On the other hand, type AB blood will be agglutinated when given to individuals with any other blood type because it includes two antigens. Therefore, blood type screening is a step commonly used in determining whether donor blood tissue is likely to be accepted or rejected by a recipient.

Another common screening procedure used is HLA (human leukocyte antigen) type matching. HLA is an antigen found on the surface most cells in the body. They make up a person's tissue type, which is different from a person's blood type. There are many HLA markers. HLA matching determines the number of HLA markers that a donor and recipient has in common. HLA matching is usually based on 10 HLA markers. The more markers the donor and recipient share, the better the match and the more likely the donor blood tissue will likely be accepted by the recipient.

In the past, bone marrow transplants and cord blood transplants were rare therapies used as a last resort. However, these therapies are becoming more and more common. As such, blood tissue cells have an increasing therapeutic value and it is important to maximize the recovery of these cells from a blood tissue sample. This is especially true when the blood tissue sample is umbilical cord blood. Cord blood can only be collected at birth from umbilical cords and have a limited volume. In other words, umbilical cord blood samples are extremely valuable and it is important to maximize recovery of therapeutic cells from these samples.

With many medical procedures, all cells from a donor blood tissue sample would be introduced into a recipient. However, only certain cells within the sample actually contribute to a therapeutic effect in the recipient. Other cells often do not contribute to the therapeutic effect and might even act against the therapeutic effect by delaying healing or causing rejection of the blood tissue. One common cell that causes rejection of donor blood tissue is the erythrocyte. For example, in the past, when giving bone marrow transplants, doctors did not take ABO blood types into consideration when doing so. However, research has since shown that the donor bone marrow has significant contamination with donor erythrocytes. These donor erythrocytes in turn can cause post-transplant complications in the recipient, such as delayed red cell engraftment, immune hemolysis, fatal hemolysis, acute GVHD, late onset hemolysis and delayed platelet engraftment. In order to address this, many transplantation procedures now require both HLA matching and ABO blood type compatibility between the donor and the recipient. The main drawback to this approach is that it further limits the number of donors and recipients that match. Other blood cells in donor blood tissue that can cause complications in recipients include granulocytes and monocytes, because these cells are pro-inflammatory.

Therefore, it would be desirable to separate and remove certain desired therapeutic cells from a blood tissue sample while removing or undesired cells from the sample. The therapeutic cells can then be inserted into recipients to achieve the desired therapeutic effect without significant contamination with undesired cells. This would allow for fewer of the undesired cells to be introduced into the recipient, thereby minimizing the undesirable effects resulting from the introduction of those cells.

Prior art methods have been used to try to remove erythrocytes cells from blood tissue samples. However, these methods need improvement because they do not completely remove erythrocytes. For example, one prior art method (known as the Rubinstein method) is used in several cord blood storage banks and commonly results in only 60-75% erythrocyte removal. It would be desirable to provide a cell separation composition and method that further maximizes the % removal of erythrocytes. This would allow for more donor and recipients to match since ABO blood type compatibility would not be as important when the % removal of erythrocytes are maximized.

It would also be desirable to provide a cell separation composition and method that further maximizes the % recovery of desired therapeutic cells. This would allow for more efficient use of valuable blood tissue samples, such as umbilical cord blood.

### SUMMARY

Certain embodiments provide a cell separation composition. The cell separation composition can be mixed with a blood tissue sample to separate therapeutic cells from undesired cells in the sample. As used herein, the term “therapeutic cells” refers to any cell in a blood tissue sample that is desired to be used in a medical procedure to achieve a therapeutic effect. Also, as used herein, the term “undesired cells” refers to any cell in a blood tissue sample that is not intended to be used in a medical procedure.

In some cases, the therapeutic cells include leukocytes. In certain cases, the therapeutic cells include lymphocytes, such as T-cells and B-cells, which can be used in immunotherapies. In other cases, the therapeutic cells include hematopoietic stem cells, which can be used to restore a hematopoietic system. In other cases, the therapeutic cells include dendritic cells, which can be used in cellular vaccinations. In yet other cases, the therapeutic cells include platelets, which can be used as a source of growth factors. In further cases, the therapeutic cells include endothelial progenitor cells, which can be used in vascular therapies. Still further, the therapeutic cells can include mesenchymal and multi-lineage stem cells for orthopedic, immune regulation and other regenerative therapies.

Also, in some cases, the undesired cells include erythrocytes. Erythrocytes are undesirable because if they are present in a blood tissue sample that is introduced into a recipient, they can trigger an immune response if the recipient does not have a compatible ABO type. In other cases, the undesired cells include monocytes. In still other cases, the undesired cells include granulocytes. Monocytes and granulocytes are undesirable because they are pro-inflammatory cells that can cause undesired inflammatory responses in a recipient.

The medical procedure can be any medical procedure known in the art. Exemplary medical procedures include but are not limited to tissue culture procedures, immunophenotypic characterization procedures, diagnostic testing procedures, purification procedures, cryogenic storage procedures, and therapeutic procedures. In certain cases, the medical procedure is a therapeutic procedure. Further, when the medical procedure is a therapeutic procedure, the therapeutic cells can be cells that cause a therapeutic effect in a recipient and the undesired cells can be cells that would not contribute towards the therapeutic effect. The determination of which cells are considered therapeutic versus undesired varies depending on the therapeutic procedure. Exemplary therapeutic procedures include but are not limited to bone marrow transplants, organ transplants and blood transfusions.

Other embodiments provide methods of separating and recovering therapeutic cells from a blood tissue sample. The method includes providing a blood tissue sample and mixing the sample with a cell separation composition to form a mixture. Once the mixture is formed, the method includes allowing the cell separation composition to separate cells in the blood tissue sample into a first partition and a second partition. The first partition includes the desired therapeutic cells and the second partition includes the undesired cells. The method then includes removing only the first partition from the mixture. The first partition can be further concentrated, cryogenically stored, introduced into a recipient or used in any other medical procedure.

In some embodiments, the cell separation composition and method are used to separate and recover all cells from a blood tissue sample except for erythrocytes. In this embodiment, the therapeutic cells include all varieties of leukocytes (i.e. all nucleated white blood cells) and platelets and the undesired cells include erythrocytes. The cell separation composition comprises, consists essentially of or consists of an erythrocyte reducing zeta-potential component, a component that chelates Ca⁺² and/or Mg⁺² and an isotonic buffered saline. In certain cases, the cell separation composition comprises, consists essentially of or consists of heta starch, EDTA and phosphate buffered saline.

In other embodiments, the cell separation composition and method are used to separate and recover all cells from a blood tissue sample except for erythrocytes, monocytes and granulocytes. In this embodiment, the therapeutic cells include lymphocytes and platelets and the undesired cells include erythrocytes, monocytes and granulocytes. The cell separation composition comprises, consists essentially of or consists of an erythrocyte reducing zeta-potential component, divalent cations, anti-CD15 antibodies and an isotonic buffered saline. In certain cases, the cell separation composition comprises, consists essentially of or consists of heta starch, Ca⁺² and/or Mg⁺² ions, anti-CD15 antibodies and an isotonic buffered saline.

### BRIEF DESCRIPTION OF THE DRAWINGS

The following drawings are illustrative of particular embodiments of the invention and therefore do not limit the scope of the invention. The drawings are not necessarily to scale (unless so stated) and are intended for use in conjunction with the explanations in the following detailed description. Embodiments of the invention will hereinafter be described in conjunction with the appended drawings, wherein like numerals denote like elements.

FIG. 1 is schematic representative of a method of separating cells in a blood tissue sample using a cell separation composition.

FIG. 2 is a hematological and flow cytometric comparison of blood tissue sample before separation, after separation using a prior art Rubinstein method, and after separation using a Formula #1 method.

FIG. 3 is a hematological and flow cytometric comparison of blood tissue sample before separation, after separation using a prior art Rubinstein method, and after separation using a Formula #2 method.

### DETAILED DESCRIPTION

The present invention relates to cell separation compositions and methods for separating and recovering desired therapeutic cells from a blood tissue sample. Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. Publications, patent applications, patents, and other references mentioned herein are incorporated by reference. In case of conflict, the present specification, including definitions, will control. In addition, the materials, methods, and examples are illustrative only and not intended to be limiting.

Compositions and Methods for Recovering All Cells Except for Erythrocytes

Some embodiments provide a cell separation composition and method that can be used to separate and recover all cells from a blood tissue sample except for erythrocytes. In this embodiment, the therapeutic cells include all varieties of leukocytes (i.e. all nucleated white blood cells) and platelets and the undesired cells include erythrocytes. In certain embodiments, the cell separating composition comprises, consists essentially of or consists of an erythrocyte reducing zeta-potential component, a chelating agent that chelates Ca⁺² and/or Mg⁺² and an isotonic buffered saline. In particular embodiments, the cell separation composition comprises, consists essentially of or consists of heta starch, EDTA and phosphate buffered saline. In one particular embodiment the cell separation composition consists only of heta starch, EDTA and phosphate buffered saline. When this cell separation composition mixes with a blood tissue sample, the composition separates the cells in the sample into two partitions. The first partition includes the desired therapeutic cells including leukocytes and platelets. The second partition includes the undesired erythrocytes.

The cell separation composition includes a component that reduces erthyrocyte zeta-potential. The erthyrocyte zeta-potential is the net negative charge on an erythrocyte cell membrane. In some cases, the erythrocyte reducing zeta-potential component is heta starch. Thus, in many embodiments the cell separation composition includes heta starch. The heta starch neutralizes negative charges on an erythrocyte cell membrane, which causes erythrocytes to aggregate.

In some cases, the heta starch is present in the composition at a concentration in the range of between about 1% to about 5%, such as between about 1.5% to about 3.0% or between about 2% to about 3%. In certain cases, the heta starch has a concentration of about 2%. In other cases, the heta starch has a concentration of about 3%. Applicant has discovered the heta starch concentrations of 2% and 3% both achieved best results when used in cell separation and removal methods. One suitable commercial source of heta starch is heta starch from B Braun Medical in Irvine, Calif., which can be provided liquid form at a concentration of 6% heta starch in physiologic saline. The heta starch can then be added to an isotonic buffered saline in an amount that provides the desired concentration of heta starch.

The cell separation composition also includes an isotonic buffered saline. In many cases, the isotonic buffered saline is a phosphate buffered saline. In certain cases, the isotonic buffered saline has the following composition: NaCl (0.137M), potassium chloride (0.0027M), sodium dibasic (0.008M) and potassium phosphate monobasic (0.002M).

The cell separation composition also includes a chelating agent that chelates Ca⁺² and/or Mg⁺². In some cases, the chelating agent is a Ca⁺² chelator. The Ca⁺² chelator prevents activation of granulocytes, which prevents them from aggregating with each other and with other leukocytes. In many cases, the Ca⁺² chelator is EDTA. The EDTA can be provided at a concentration in the range of between about 0.1 mM to about 50 mM. In some cases, the EDTA can be provided at a concentration in a range of between about 0.1 mM to about 10 mM. One suitable commercial source of EDTA is EDTA from Sigma Chemical in St. Louis, Mo. The EDTA can be provided in powdered form and added to the liquid composition including an isotonic buffered saline and heta starch.

One particular embodiment of a cell separation composition is shown in Table 1 below and is referred to herein as “Formula #1.” Formula #1 is a composition that separates erythrocytes from all other cells in a blood tissue sample. Formula #1 can be used when it is desired to maximize erythrocyte removal and to maximize recovery of all leukocytes and platelets from a blood tissue sample. For certain therapeutic procedures, is often desired to recover all varieties of leukocytes (i.e. all nucleated white blood cells) and platelets, particularly when the blood tissue sample is umbilical cord blood. Umbilical cord blood is often cryogenically preserved and the later usability of the blood depends on the total nucleated cellularity of the nucleated white blood cells.

TABLE 1
Cell Separation Composition Formula #1

| Component | Amount | | Concentration (before mixing/as added) |
|---|---|---|---|
| Heta Starch | 333 | ml | 6% |
| Phosphate | 667 | ml | NaCl (.137M), potassium chloride |
| Buffered | | | (.0027M), sodium dibasic (.008M) and |
| Saline | | | potassium phosphate monobasic (.002M) |
| EDTA | 0.404 | g/L | 1 mM |

Other embodiments provide methods of separating and recovering all blood cells other than erythrocytes from a blood tissue sample. The method includes providing a blood tissue sample and mixing the sample with a cell separation composition to form a mixture. The cell separation composition can be any of the cell separation compositions described above that separate and recover all blood cells other than erythrocytes from a blood tissue sample. The blood tissue sample can include any desired blood tissue. In some cases, the blood tissue sample includes blood tissue selected from the group consisting of peripheral blood, umbilical cord blood, menstrual blood, bone marrow, spleen tissue and lymphatic tissue. In certain cases, the spleen tissue is disaggregated spleen tissue and the lymphatic tissue is disaggregated lymphatic tissue.

The cell separation composition can also be mixed with the blood tissue sample according to a specific cell separation composition/blood tissue sample ratio. In some cases, the ratio is in a range of between about 1:2 to about 10:1. In certain cases, the ratio is in a range of between about 1:1 to about 2:1. In particular cases, the ratio is about 3:2. In certain particular cases, the blood tissue sample includes peripheral blood, umbilical cord blood or bone marrow and the ratio is about 3:2. The ratio selected can also depend on the concentration of specific components in the cell separation composition and also on the type of blood tissue sample.

Once the mixture is formed, the method includes allowing the cell separation composition to separate cells in the blood tissue sample into a first partition and a second partition. The first partition includes the desired therapeutic leukocytes and platelets and the second partition includes the undesired erythrocytes. The method then includes recovering only the second partition from the mixture. The recovered second partition now includes the desired therapeutic leukocytes and platelets, which can be further concentrated, cryogenically stored, introduced into a recipient or used in any other medical procedure.

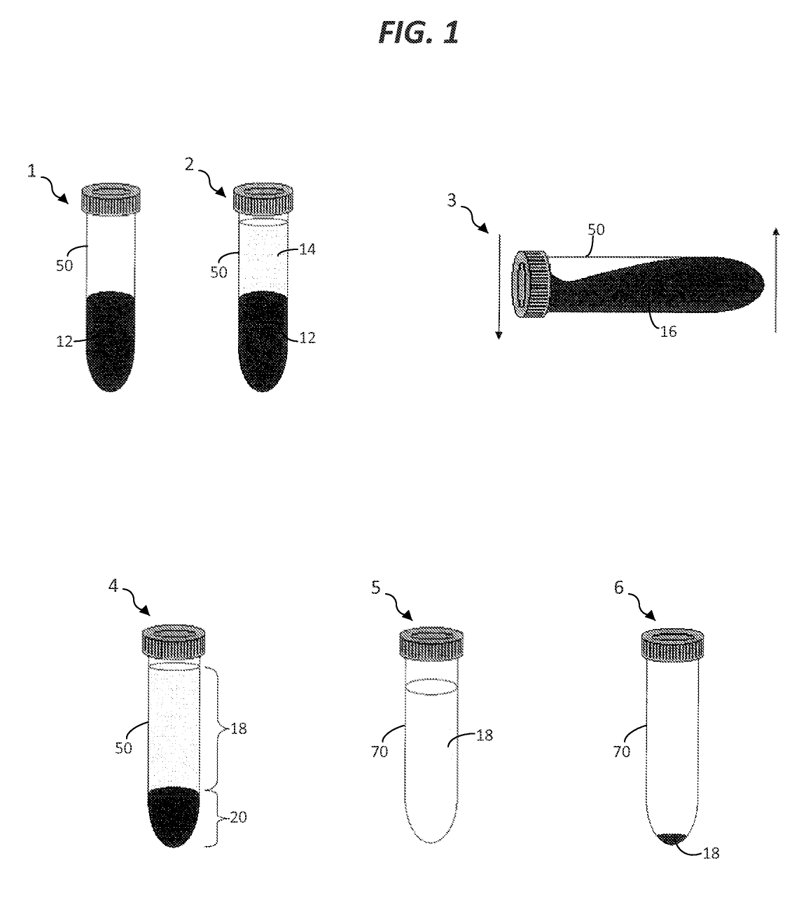

FIG. 1 illustrates an embodiment of a method 10 of separating and recovering all blood cells other than erythrocytes from a blood tissue sample. The method 10 includes a first step 1 that includes adding a blood tissue sample 12 to a sterile container 50. The blood tissue sample can be any sample according to any of the embodiments already described.

Step 2 includes adding a cell separation composition 14 to the container 50. The cell separation composition 14 comprises, consists essentially of or consists of an erythrocyte reducing zeta-potential component (e.g., heta starch), a chelating agent that chelates Ca⁺² and/or Mg⁺² (e.g., EDTA) and an isotonic buffered saline. The cell separation composition 14 can also be added in an amount that provides a cell separation composition/blood tissue sample ratio according to any of the embodiments already described. In some cases, the cell separation composition 14 is added at a ratio of 3:2.

Step 3 includes moving the container 50 such that the blood tissue sample 12 and the cell separation composition 14 mix together to form a mixture 16. The container 50 can be shaken, vibrated, rocked or moved any desired motion to mix the mixture 16. In certain cases, the container 50 is positioned on a rocker platform, which provides rocking motion to the container 50. The container 50 can also be subjected to motion for a period of time. For example, the container 50 can be subjected to motion for a time period in the range of between about 1 minute and about 1 hour. In some cases, the container 50 is subjected to motion for about 1 minute.

Step 4 includes providing the container 50 in a stationary position for a period of time to allow for cell separation in the blood tissue sample 12 to take place. In certain cases, the stationary position is a stationary upright position. In some cases, the container 50 is held in a stationary position for a period of time in the range of between about 1 minute and about 1 hour. In certain cases, the period of time is about 30 minutes.

After the period of time expires, the mixture 16 will have separated into a first partition 18 and a second partition 20. The first partition 18 and the second partition 20 will be separated be a well delineated demarcation between them. The first partition 18 includes the desired therapeutic leukocyte cells and platelets and the second partition 20 includes the undesired erythrocytes. The first partition 18 includes at least about 90% of leukocytes from the blood tissue sample. Likewise, the second partition includes at least about 97% of erythrocytes from the blood tissue sample.

During cell separation, the erythrocyte reducing zeta-potential component (e.g., heta starch) neutralizes negative charges on erythrocyte cell membranes in the mixture 16 the. This causes the erythrocytes to form structures or aggregates resembling stacked coins called rouleaux. These structures have a high sedimentation rate in comparison to single cells in suspension. The structures quickly settle, falling to the bottom of the container to become part of the second partition 20.

Also, the chelating agent (e.g., EDTA) prevents activation of granulocytes. When granulocytes are activated, they have elevated expression of cell surface adhesion molecules that causes granulocytes to bind to other granulocytes and to other leukocytes to form aggregates. Since the granulocytes are not activated, they do not bind to other granulocytes and to other leukocytes to form aggregates. As such, the granulocytes and leukocytes do not aggregate and fall to the bottom of the container. They instead remain in suspension as part of the first partition 18.

At step 5, the first partition 18 is recovered from the container 50 and inserted into a new container 70. This step can be accomplished using any removal or recovery method known in the art, such as removing using a pipette. The second partition 20, which includes the undesired erythrocytes, is discarded.

At step 6, the first partition 18 is subjected to a concentration method to concentrate the desired cells. In some cases, the concentration method is a centrifugation method. The concentrated desired cells include at least about 90% of leukocytes from the blood tissue sample and are then ready to be used in a medical procedure.

The cell separation composition and method in this embodiment drastically improves leukocyte recovery and erythrocyte removal over prior art methods, such as the Rubinstein method. In fact, the Rubinstein method yields an average leukocyte recovery of 80% from a blood tissue sample whereas the present method and composition recovers at least about 90% of leukocytes from a blood tissue sample. Also, the Rubinstein method yields an average erythrocyte removal of about 70% whereas the present method and composition removes at least about 97% of erythrocytes. Additionally, the Rubinstein method yields an average lymphocyte recovery of 80% whereas the present method and composition recovers at least about 95% of lymphocytes.

### EXAMPLE 1

Five peripheral blood tissue samples were collected. Portions of these samples were not subjected to cell separation and were set aside. Other portions of the samples were subjected to cell separation using a prior art Rubinstein method. Each sample was first added to a sterile container. Rubinstein formula was next added to the container at a Rubinstein formula/peripheral blood ratio of 5:2 to form a mixture. The mixtures were then centrifuged at 50×g for 10 minutes to separate the cells into a supernatant and an erythrocyte pellet. The supernatant and top 20% of the erythrocyte pellet were removed by pipette and centrifuged at 300×g for 6 minutes to concentrate the cells. This method is referred to herein as the “Rubinstein method.”

Other portions of the samples were subjected to cell separation using Formula 1 as the cell separation medium. Each sample was first added to a sterile container. Formula 1 was next added to the container at a Formula 1/peripheral blood ratio of 3:2 to form a mixture. The container was positioned on a rocker platform and mixed for 1 minute. Next, the container was placed in an upright position for 30 minutes, during which cell separation took place to form a first partition including leukocytes and platelets and a second partition including erythrocytes. The first partition was then removed using a pipette and inserted into a new sterile container. The first partition in the new sterile container was concentrated by centrifugation at 300×g for 6 minutes. This method is referred to herein as the “Formula #1 method.”

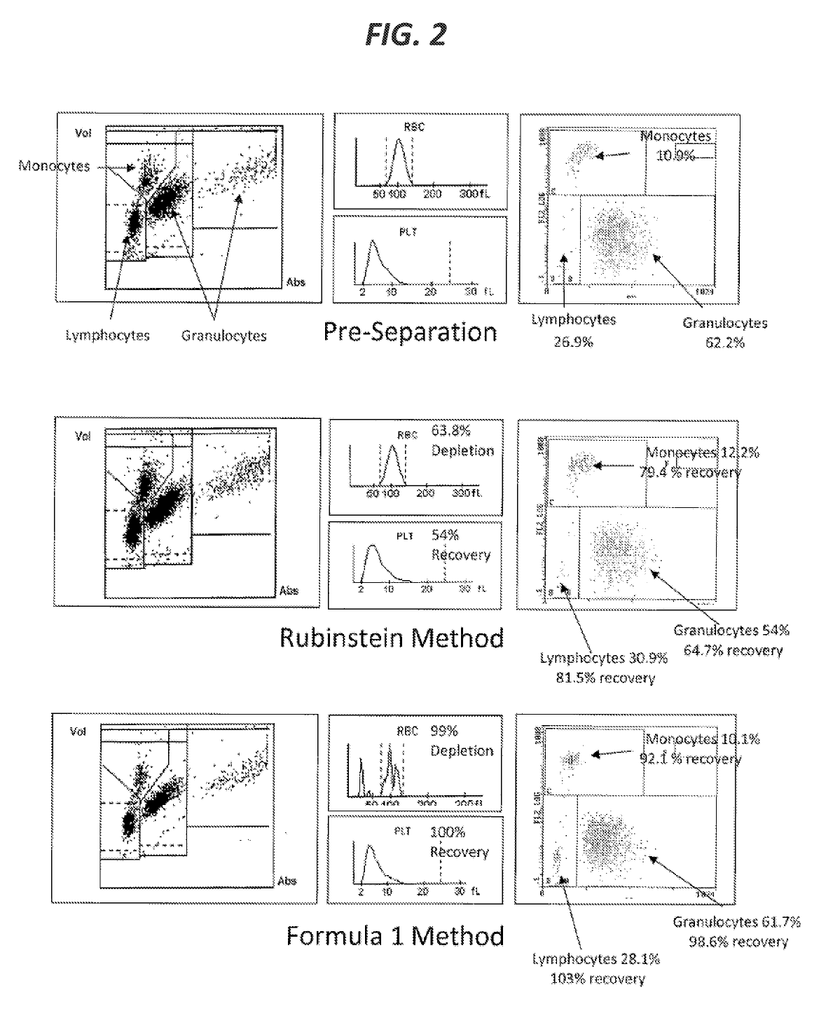

The pre-separation sample, the sample separated using the Rubinstein method and the sample separated using the Formula #1 method were each analyzed by a Beckman Coulter AcT 5diff CP hematology analyzer. The samples were also each analyzed by a Coulter Epics XL flow cytometer. FIG. 2 shows hematology histograms and flow cytometry histograms obtained for one sample. As shown, in this sample, the Rubinstein method depleted far less erythrocytes from the sample than the Formula #1 method. Also, the Rubinstein method recovered far less platelets and leukocytes than the Formula #1 method.

Table 2 below also compares the % recoveries of leukocytes and platelets and % depletion of erythrocytes for all five samples separated with the Rubinstein method and with the Formula #1 method.

TABLE 2

| | Leukocyte | Platelet | Erythrocyte |
|---|---|---|---|
| Sample | Recovery % | Recovery % | Depletion % |
| Formula #1 | 98.8 ± 1.82% | 99.52 ± 5.12% | 98.94 ± 0.2% |
| Method | | | |
| Rubinstein | 79.01 ± 10.39% | 64.3 ± 6.38% | 66.41 ± 2.81% |
| Method | | | |

As shown, cell separation using the Formula #1 method recovered a much higher percentage of leukocytes and platelets than cell separation using the Rubinstein method. In fact, the Formula #1 method recovered at least about 97% of leukocytes in a blood tissue sample. Also, the Formula #1 method recovered at least about 94% of platelets in a blood tissue sample. Likewise, the Formula #1 method allowed for a higher erythrocyte depletion than obtained with the Rubinstein method. In fact, the Formula #1 method depleted at least about 98% of erythrocytes in a blood tissue sample.

Compositions and Methods for Recovering All Cells Except for Erythroctyes, Monocytes and Granulocytes

Some embodiments provide a cell separation composition and method that can be used to separate and recover all cells from a blood tissue sample except for erythrocytes, monocytes and granulocytes. In this embodiment, the therapeutic cells include lymphocytes and platelets and the undesired cells include erythrocytes, monocytes and granulocytes. In certain embodiments, the cell separation composition comprises, consists essentially of or consists of an erythrocyte reducing zeta-potential component, divalent cations, anti-CD15 antibodies and an isotonic buffered saline. In one embodiment, the cell separation composition consists only of an erythrocyte reducing zeta-potential component, divalent cations, anti-CD15 antibodies and an isotonic buffered saline. When this cell separation composition is mixed with a blood tissue sample, the composition separates the cells in the sample into two partitions. The first partition includes the desired therapeutic cells including lymphocytes and platelets. The second partition includes the undesired erythrocytes, monocytes and granulocytes.

In many cases, the erythrocyte reducing zeta-potential component in this composition is heta starch. In some cases, the heta starch is present in the composition at a concentration in the range of between about 1% to about 5%, such as between about 1.5% to about 3.0% or between about 2% to about 3%. In certain cases, the heta starch has a concentration of about 2%. In other cases, the heta starch has a concentration of about 3%. Again, Applicant has discovered the heta starch concentrations of 2% and 3% both achieved best results when used in cell separation and recovery methods.

Also, in many cases, the divalent cations are Ca⁺² and/or Mg⁺² ions. Ca⁺² and/or Mg⁺² ions can be provided in the form of a balanced salt solution, such as Hank's balanced salt solution.

Finally, the anti-CD15 antibodies can include any anti-CD15 antibody that does not react with monocytes. Exemplary anti-CD15 antibodies include, but are not limited to, AHN1.1 (murine IgM isotype), FMC-10 (murine IgM isotype), BU-28 (murine IgM isotype), MEM-157 (murine IgM isotype), MEM-158 (murine IgM isotype), MEM-167 (murine IgM isotype) and 324.3.B9 (murine IgM isotype). In some cases, the anti-CD 15 antibodies are present in the composition at a concentration in the range of between about 0.01 mg/L to about 15 mg/L, such as between about 0.1 mg/L to about 15 mg/L, between about 0.1 mg/L to about 10 mg/L, or between about 1 mg/L to about 5 mg/L. In certain cases, the antibodies have a concentration of about 1 mg/L.

During cell separation, the anti-CD15 antibodies bind to CD15 antigens on cell surfaces of granulocytes. This activates the granulocytes and stimulates the expression of a variety of adhesion molecules such as lymphocyte function-associated antigen-1, CD11a/CD18 (LFA-1) and intracellular adhesion molecule-1, CD54 (ICAM-1). These adhesion molecules cause granulocytes to bind to other granulocytes and to monocytes. The divalent cations enable the granulocytes to be activated and then express adhesion molecules.

One particular embodiment of a cell separation composition is shown in Table 3 below and is referred to herein as “Formula #2.” Formula #2 is a composition that separates erythrocytes, granulocytes and monocytes from all other cells in a blood tissue sample. Formula #2 is used when it is desired to maximize erythrocyte, granulocyte and monocyte removal from a blood tissue sample.

TABLE 3
Cell Separation Composition (Formula #2)

| | | | Concentration(before |
|---|---|---|---|
| Component | Amount | | mixing, as added) |
| Heta Starch | 333 | ml | 6% |
| Hanks buffered saline | 667 | ml | 1x |
| Anti-CD15 antibody | 0.1-0.2 | mg/L | N/A |
| (murine IgM monoclonal | | | |
| antibody clone 324.3.B9) | | | |

Other embodiments provide methods of separating and recovering lymphocytes and platelets from a blood tissue sample while removing erythrocytes, monocytes and granulocytes. The method includes providing a blood tissue sample and mixing the sample with a cell separation composition to form a mixture. The cell separation composition can be any of the cell separation compositions described above that separate and recover all blood cells except for erythrocytes, monocytes and granulocytes. The blood tissue sample can include any desired blood tissue. In some cases, the blood tissue sample includes blood tissue selected from the group consisting of peripheral blood, umbilical cord blood, menstrual blood, bone marrow, spleen tissue and lymphatic tissue. In certain cases, the spleen tissue is disaggregated spleen tissue and the lymphatic tissue is disaggregated lymphatic tissue.

The cell separation composition can also be mixed with the blood tissue sample according to a specific cell separation composition/blood tissue sample ratio. In some cases, the ratio is in a range of between about 1:2 to about 10:1. In certain cases, the ratio is in a range of between about 1:1 to about 2:1. In particular cases, the ratio is about 3:2. In certain particular cases, the blood tissue sample includes peripheral blood, umbilical cord blood or bone marrow and the ratio is about 3:2. The ratio selected can also depend on the concentration of specific components in the cell separation composition and also on the type of blood tissue sample.

Once the mixture is formed, the method includes allowing the cell separation composition to separate cells in the blood tissue sample into a first partition and a second partition. The first partition includes the desired therapeutic lymphocytes and platelets and the second partition includes the undesired erythrocytes, monocytes and granulocytes. The method then includes recovering only the second partition from the mixture. The second partition now includes the desired therapeutic lymphocytes and platelets, which can be further concentrated, cryogenically stored, introduced into a recipient or used in any other medical procedure.

Referring back to FIG. 1, another embodiment of a method 10 of separating and recovering all blood cells other than erythrocytes from a blood tissue sample will be described. The method 10 includes a step 1 of adding a blood tissue sample 12 to a sterile container 50 and a step 2 of adding a cell separation composition 14 to the container 50. The cell separation composition 14 comprises, consists essentially of or consists of an erythrocyte reducing zeta-potential component, divalent cations, anti-CD15 antibodies and an isotonic buffered saline. The cell separation composition 14 can also be added in an amount that provides a cell separation composition/blood tissue sample ratio according to any of the embodiments already described. In some cases, the cell separation composition 14 is added at a ratio of 3:2.

Step 3 includes moving the container 50 such that the blood tissue sample 12 and the cell separation composition 14 mix together to form a mixture 16. The container 50 is also subjected to motion for a period of time in the range of between about 1 minute and about 1 hour. In some cases, the container 50 is subjected to motion for about 30 minutes.

Step 4 includes providing the container 50 in a stationary position for a period of time to allow for cell separation in the blood tissue sample 12 to take place. In certain cases, the stationary position is a stationary upright position. In some cases, the container 50 is held in a stationary position for a period of time in the range of between about 1 minute and about 1 hour. In certain cases, the period of time is about 30 minutes.

After the period of time expires, the mixture 16 will have separated into a first partition 18 and a second partition 20. The first partition 18 includes the desired therapeutic cells including lymphocyte cells and platelets and the second partition 20 includes the undesired erythrocytes, monocytes and granulocytes. The first partition 18 includes at least about 90% lymphocytes from the blood tissue sample. Likewise, the second partition 20 includes at least about 97% erythrocytes from the sample.

During cell separation, the erythrocyte reducing zeta-potential component (e.g., heta starch) neutralizes negative charges on erythrocyte cell membranes in the mixture 16 and causes the erythrocytes to aggregate and fall to the bottom of the container to become part of the second partition 20. Also, the anti-CD15 antibodies bind to CD15 antigens on cell surfaces of granulocytes, which causes granulocytes to express adhesion molecules that cause granulocytes to bind to other granulocytes and to monocytes. Thus, the granulocytes and monocytes also aggregate and fall to the bottom of the container to become part of the second partition 20.

At step 5, the first partition 18 is recovered from the container 50 and inserted into a new container 70. This step can be accomplished using any removal or recovery method known in the art, such as removing using a pipette. The second partition 20, which includes the undesired erythrocytes, granulocytes and monocytes, is discarded.

At step 6, the first partition 18 is subjected to a concentration method to concentrate the desired therapeutic cells including lymphocytes and platelets. In some cases, the concentration method is a centrifugation method. The concentrated cells include at least about 90% of lymphocytes from the blood tissue sample and are then ready to be used in a medical procedure.

The cell composition and method in this embodiment improves lymphocyte recovery and erythrocyte, monocyte and granulocyte removal over prior art methods. In fact, the Rubinstein method yields an average lymphocyte recovery of 80% from a blood tissue sample whereas the present method and composition recovers at least about 90% lymphocytes from a blood tissue sample. Also, the Rubinstein method yields an average erythrocyte removal of about 70% whereas the present method and composition removes at least about 97% erythrocytes from a blood tissue sample. Further, the Rubinstein method yields an average monocyte removal of about 20% whereas the present method and composition removes at least about 95% monocytes from a blood tissue sample. Finally, the Rubinstein method yields an average granulocyte removal of about 35% whereas the present method and composition removes at least about 95% granulocytes from a blood tissue sample.

EXAMPLE 2

Five peripheral blood tissue samples were collected. Portions of these samples were not subjected to cell separation and were set aside. Other portions of the samples were subjected to cell separation using the Rubinstein method defined above in Example 1. Other portions of the samples were subjected to cell separation using Formula 2 as the cell separation medium. Each sample was first added to a sterile container. Formula 2 was next added to the container at a Formula 2/peripheral blood ratio of 3:2 to form a mixture. The container was positioned on a rocker platform and mixed for 30 minutes. Next, the container was placed in an upright position for 30 minutes, during which cell separation took place to form a first partition including lymphocytes, stem cells and platelets and a second partition including erythrocytes, granulocytes and monocytes. The first partition was then removed using a pipette and inserted into a new sterile container. The first partition in the new sterile container was concentrated by centrifugation at 300×g for 6 minutes. This method is referred to herein as the “Formula #2 method.”

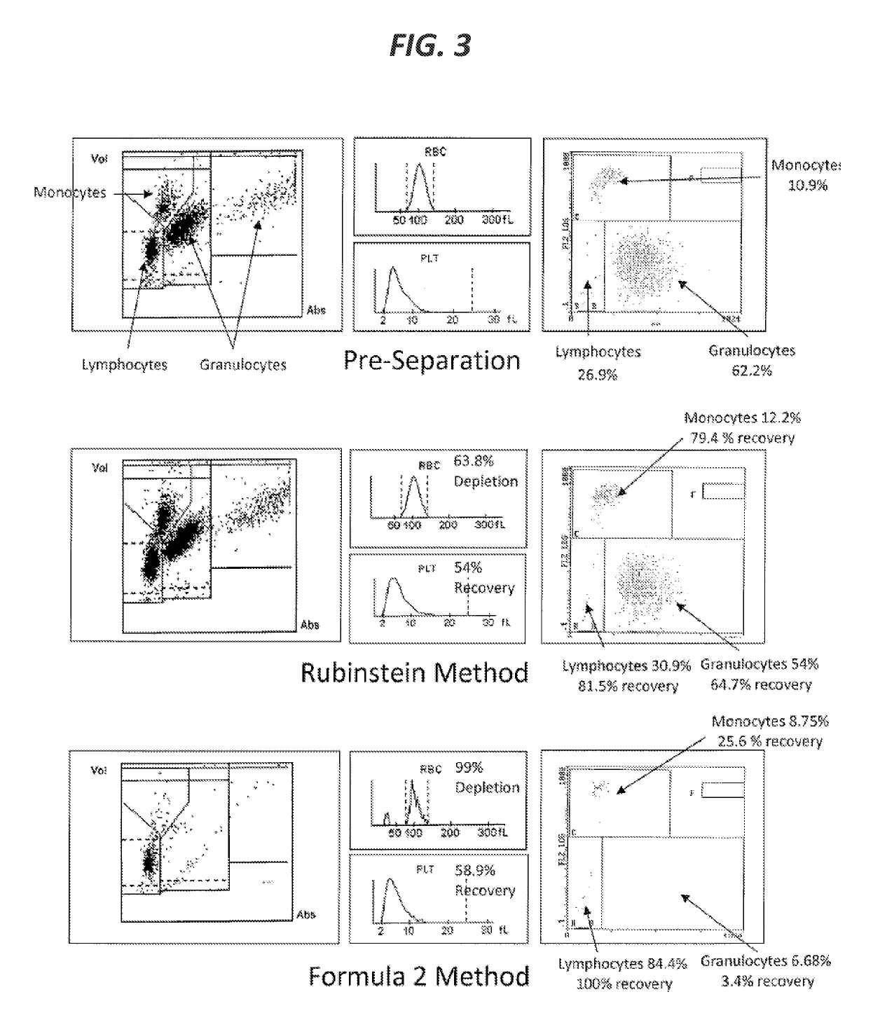

The pre-separation sample, the sample separated using the Rubinstein method and the sample separated using the Formula #2 method were each analyzed by a Beckman Coulter AcT 5diff CP hematology analyzer. The samples were also each analyzed by a Coulter Epics XL flow cytometer. FIG. 3 shows hematology histograms and flow cytometry histograms obtained for one sample. As shown, in this sample, the Rubinstein method depleted far less erythrocytes, granulocytes and monocytes from the sample than the Formula #2 method. Also, the Rubinstein method recovered far less lymphocytes than the Formula #2 method.

Table 4 below also compares the % recovery of lymphocytes and % depletion of erythrocytes, monocytes and granulocytes for all five samples separated with the Rubinstein method and all samples separated with the Formula #1 method.

TABLE 4

| | Lymphocyte | Monocyte | Granulocyte | CD3 + | |
|---|---|---|---|---|---|
| Sample | Recovery % | Recovery % | Recovery | T cells | Hematocrit |
| Formula #1 | 104.57 ± 2.59% | 99.66 ± 13.79% | 96.92 ± 2.35% | 104.46 ± 3.84% | 0 |
| Method | | | | | |
| Formula #2 | 101.11 ± 1.97% | 42.03 ± 22.76% | 4.73 ± 6.02% | 108.09 ± 3.23% | 0 |
| Method | | | | | |
| Rubinstein | 89.64 ± 8.80% | 90.55 ± 15.05% | 72.60 ± 11.02% | 89.22 ± 7.41% | 24.86 ± 2.71 |
| Method | | | | | |

As shown, cell separation using the Formula #1 method recovered a much higher percentage of lymphocytes, monocytes, granulocytes, and CD3 + T cells than what was recovered using the Rubinstein method. In fact, the Formula #1 method recovered at least about 98% of lymphocytes in the samples, at least about 86% of monocytes, at least about 93% granulocytes and at least about 98% CD3 + T cells.

Also, as shown, cell separation using the Formula #2 method recovered a much higher percentage of lymphocytes and CD3+ T cells than cell separation using the Rubinstein method. In fact, the Formula #2 method recovered at least about 98% of lymphocytes in the samples and at least about 98% CD3+ T cells. Also, the Formula #2 method depleted a higher percentage of monocytes and granulocytes in the samples than what was recovered using the Rubinstein method. In fact, the Formula #2 method depleted at least about95% of monocytes and at least about 95% granulocytes in the samples.

What is claimed is:

1. A cell separation composition for removing therapeutic cells from a blood tissue sample, the composition consisting essentially of heta starch, phosphate buffered saline, and EDTA.

2. The composition of claim 1 wherein the heta starch is present in the composition at a concentration in a range of between of about 1.5% to 3.0%.

3. The composition of claim 1 wherein the EDTA is present in the composition at a concentration in a range of between of about 0.1 mM to about 10 mM.

4. A cell separation composition for removing therapeutic cells from a blood tissue sample, the composition consisting essentially of: heta starch, buffered physiologic saline, Ca⁺² ions and/or Mg⁺² ions, and anti-CD15 antibodies.

5. The composition of claim 4 wherein the heta starch is present in the composition at a concentration in a range of between of about 1.5% to 3.0%.

6. The composition of claim 4 wherein the anti-CD15 antibodies are present in the composition at a concentration in a range of between of about 0.001 mg/L to about 15 mg/L.

7. The composition of claim 4 wherein the Ca⁺² ions and/or Mg⁺² ions are present in the composition at a concentration in a range of between of about 0.1 mM to about 10 mM.

8. A method for separating and recovering therapeutic cells from a blood tissue, the method comprising:
providing a blood tissue sample;
mixing the blood tissue sample with a cell separation composition to form a mixture, the cell separating composition consisting essentially of heta starch, phosphate buffered saline, and EDTA;
allowing the mixture to separate into a first partition and a second partition, wherein the first partition includes therapeutic cells and the second partition includes undesired cells; and
removing only the first partition from the mixture.

9. The method of claim 8 wherein the undesired cells include erythrocytes and the second partition includes at least about 97% of erythrocytes present in the blood tissue sample.

10. The method of claim 8 wherein the therapeutic cells include leukocytes and the first partition includes at least about 90% of leukocytes present in the blood sample.

11. The method of claim 8 wherein the therapeutic cells include platelets and the first partition includes at least about 90% of platelets present in the blood sample.

12. The method of claim 8 wherein the step of contacting the blood tissue with a cell separation composition comprises providing the cell separation composition in a cell separation composition/blood tissue ratio of about 3:2.

13. The method of claim 8 wherein the blood tissue sample includes blood tissue selected from the group consisting of peripheral blood, umbilical cord blood, menstrual blood, bone marrow, spleen tissue and lymphatic tissue.

14. The method of claim 8 wherein the heta starch is present in the cell separation composition at a concentration in a range of between about 1.5% to 3.0%.

15. The method of claim 8 wherein the EDTA is present in the cell separation composition at a concentration in a range of between about 0.1 mM to about 10 mM.

16. A method for removing therapeutic cells from a blood tissue, the method comprising:
providing a blood tissue sample;
mixing the blood tissue sample with a cell separating composition to form a mixture, the cell separating composition comprising heta starch, buffered physiologic saline, Ca⁺² ions, Mg⁺² ions, and anti-CD15 antibodies;
allowing the mixture to separate into a first partition and a second partition, wherein the first partition includes therapeutic cells and the second partition includes undesired cells; and
removing only the first partition from the mixture.

17. The method of claim 16 wherein the undesired cells include erythrocytes and the second partition includes at least about 97% of erythrocytes present in the blood tissue sample.

18. The method of claim 16 wherein the undesired cells include monocytes and the second partition includes at least about 95% of monocytes present in the blood tissue sample.

19. The method of claim 16 wherein the undesired cells include granulocytes and the second partition includes at least about 95% of granulocytes present in the blood tissue sample.

20. The method of claim 16 wherein the therapeutic cells include lymphocytes and the first partition includes at least about 98% of lymphocytes present in the blood sample.

21. The method of claim 16 wherein the therapeutic cells include platelets and the first partition includes at least about 80% of platelets present in the blood tissue sample.

22. The method of claim 16 wherein the step of contacting the blood tissue with a cell separation composition comprises providing the cell separation composition in a cell separation composition/blood tissue ratio of about 3:2.

23. The method of claim 16 wherein the blood tissue sample includes blood tissue selected from the group consisting of peripheral blood, umbilical cord blood, menstrual blood, bone marrow, spleen tissue and lymphatic tissue.

24. The method of claim 16 wherein the heta starch is present in the cell separation composition at a concentration in a range of between about 1.5% to 3.0%.

25. The method of claim 16 wherein the anti-CD15 antibodies are present in the cell separation composition at a concentration in a range of between about 0.001 mg/L to about 15 mg/L.

26. The method of claim 16 wherein the Ca⁺² ions and/or Mg⁺² ions are present in the cell separation composition at a concentration in a range of between about 0.1 μM to about 10 μM.
